(19) United States
(12) Patent Application Publication
(10) Pub. No.: US 2005/0010098 A1
(43) Pub. Date: Jan. 13, 2005

(54) Method and apparatus for knowledge based diagnostic imaging

(76) Inventors: Sigmund Frigstad, Trondheim (NO); Bjorn Olstad, Stathelle (NO)

Correspondence Address: Dean D. Small;Armstrong Teasdale LLP, Suite 2600, One Metropolitan Square, St. Louis, MO, 63102, US

(21) Appl. No.: 10/810,132

(22) Filed: Mar. 26, 2004

Related U.S. Application Data
(60) Provisional application No. 60/462,012, filed on Apr. 11, 2003.

(51) Int. Cl. A61B 5/05; G06F 17/00

(52) U.S. Cl. 600/407; 128/924; 706/924; 600/437; 706/50

(57) ABSTRACT

A knowledge based diagnostic imaging system, comprising diagnostic equipment for analyzing a patient to obtain a new patient data set containing at least one of MR data, CT data, ultrasound data, x-ray data, SPECT data and PET data. The diagnostic equipment automatically analyzes the new patient data set with respect to a physiologic parameter of the patient to obtain a patient value for said physiologic parameter. A database containing past patient data sets for previously analyzed patients. The past patient data sets contain data indicative of the physiologic parameter with respect to previously analyzed patients. A network interconnects the diagnostic equipment and the database to support access to the past patient data sets.

### RELATED APPLICATION

The present application relates to and claims priority from Provisional Application Ser. No. 60/462,012, filed Apr. 11, 2003, titled “Method and Apparatus for Knowledge Based Diagnostic Imaging”, the complete subject matter of which is hereby expressly incorporated in its entirety.

### BACKGROUND OF THE INVENTION

Today a wide variety of medical diagnostic imaging systems are offered to assist physicians in detecting and diagnosing pathologies. Examples of modalities that offer such diagnostic systems include ultrasound, CT, MR, PET, SPECT and x-ray, as well as mammography and the like. These diagnostic imaging systems are quite specialized and may be quite expensive. Due to the nature of each system, technicians, physicians and operators typically expend a significant amount of time in learning how to operate the equipment and interpret images obtained with the equipment. Specialists may operate the equipment or interpret the resulting images. Hence, not every hospital is able to justify the expense associated with the equipment and the staff/operators that use the equipment. Also, even when a hospital offers the imaging equipment, the hospital may be unable to justify multiple staff or physicians who are specially trained to utilize the equipment. Hence, only a few doctors, technicians and operators may be fully trained on the equipment at any single hospital. This limitation in resources often creates a bottleneck for the use of the equipment and patients are not able to receive immediate examination with such equipment.

In addition, in present healthcare systems around the world, patients typically visit primary healthcare providers first, before receiving a referral to another doctor who specializes in a particular procedure and/or conducts certain types of examinations that use medical diagnostic equipment. Typically, the patient is not examined with the diagnostic equipment until the second or third visit to a physician, as the first visit is to the primary healthcare provider. Primary healthcare providers today do not utilize diagnostic imaging equipment as part of their normal examination process. This is due in part to a lack of familiarity and training with such equipment. Consequently, primary healthcare providers are unable to apply diagnostic imaging in their diagnosis and examinations. Heretofore, unless the primary healthcare provider has received the particular specialized training needed to utilize diagnostic equipment, the existing healthcare system was unable to provide adequate quality assurance that the primary healthcare provider would properly diagnose a given pathology when viewing the diagnostic images. There has been no mechanism to educate or share knowledge with the primary healthcare providers that would facilitate such quality assurance.

One consequence of the existing healthcare system is that disease detection and treatment is forgone or delayed where it might otherwise might be obtained earlier based on closer and more frequent patient monitoring through the use of diagnostic equipment. Existing systems have been unable to provide sufficiently objective and accurate imaging methodologies to support the use of diagnostic imaging equipment by non-specialists.

A need exists for an improved infrastructure for medical imaging, and for evolving medical communications and data management systems and standards that support on-line guidance and remote off-line expert analysis of diagnostic images. A need exists for a system that supports high quality, easy to use portable scanners having automated features to achieve disease detection and that incorporate new imaging and parameter identification measurement and analysis methodologies.

### BRIEF SUMMARY OF THE INVENTION

Certain embodiments of the present invention are directed to knowledge-based diagnostic methods and apparatus that afford a new approach to primary, healthcare (HC) workflow for new patients. The first HC provider that examines each patient is able to utilize diagnostic imaging equipment to provide a more qualified initial diagnosis of the patient. In one application, low-cost, portable, high-image quality diagnostic equipment may be provided to each healthcare provider for use, early and often, during initial patient examinations. Examples of such equipment are ultrasound or x-ray equipment. While MR, CT and PET equipment is more expensive, such equipment may equally be used in the knowledge-based diagnostic methods described herein.

### BRIEF DESCRIPTION OF THE DRAWINGS

The foregoing summary, as well as the following detailed description of the embodiments of the present invention, will be better understood when read in conjunction with the appended drawings. It should be understood, however, that the present invention is not limted to the arrangements and instrumentality shown in the attached drawings.

FIG. 1 illustrates a block diagram of an ultrasound system formed in accordance with an embodiment of the present invention.

FIG. 2 illustrates a block diagram of a second ultrasound system formed in accordance with one embodiment of the present invention.

FIG. 3 illustrates an isometric drawing of a rendering box formed in accordance with one embodiment of the present invention.

FIG. 4 illustrates a healthcare network formed in accordance with an embodiment of the present invention.

FIG. 5 illustrates a healthcare network formed in accordance with an alternative embodiment of the present invention.

FIG. 6 illustrates a flow chart for a method for automatically analyzing patient data sets in accordance with an embodiment of the present invention.

### DETAILED DESCRIPTION OF THE INVENTION

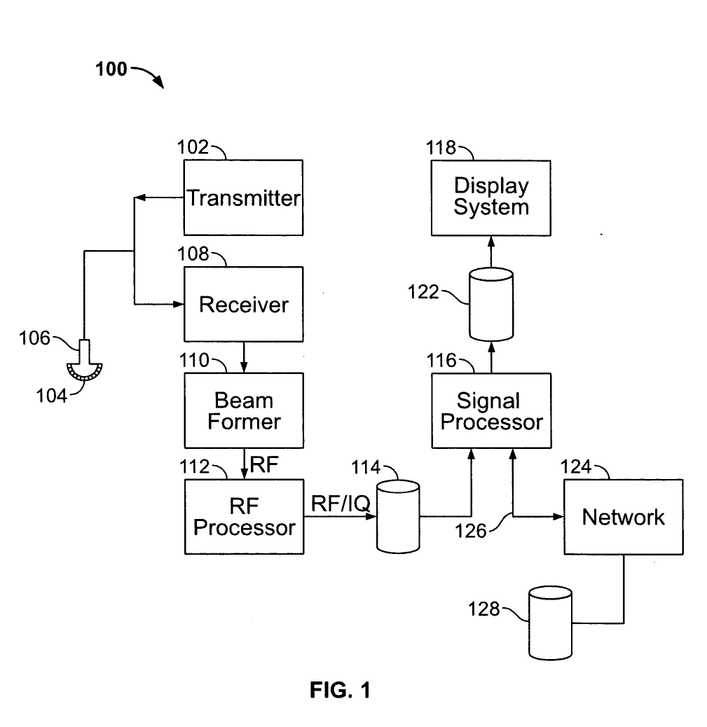

FIG. 1 illustrates a block diagram of an ultrasound system 100 formed in accordance with an embodiment of the present invention. The ultrasound system 100 includes a transmitter 102 which drives transducers 104 within a probe 106 to emit pulsed signals that are back-scattered from structures in the body, like blood cells or muscular tissue, to produce echoes which return to the transducers 104. The echoes are received by a receiver 108. The received echoes are passed through a beamformer 110, which performs beamforming and outputs an RF signal. The RF signal then passes through an RF processor 112. Alternatively, the RF processor 112 may include a complex demodulator (not shown) that demodulates the RF signal to form IQ data pairs representative of the echo signals. The RF signal or IQ data pairs may then be routed directly to RF/IQ buffer 114 for temporary storage.

The ultrasound system 100 also includes a signal processor 116 to process the acquired ultrasound information (i.e., RF signal data or IQ data pairs) and prepare frames of ultrasound information for display on display system 118. The signal processor 116 is adapted to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in RF/IQ buffer 114 during a scanning session and processed in less than real-time in a live or off-line operation.

The ultrasound system 100 may continuously acquire ultrasound information at a frame rate that exceeds 50 frames per second—the approximate perception rate of the human eye. The acquired ultrasound information is displayed on the display system 118 at a slower frame-rate. An image buffer 122 is included for storing processed frames of acquired ultrasound information that are not scheduled to be displayed immediately. Preferably, the image buffer 122 is of sufficient capacity to store at least several seconds worth of frames of ultrasound information. The frames of ultrasound information are stored in a manner to facilitate retrieval thereof according to its order or time of acquisition. The image buffer 122 may comprise any known data storage medium.

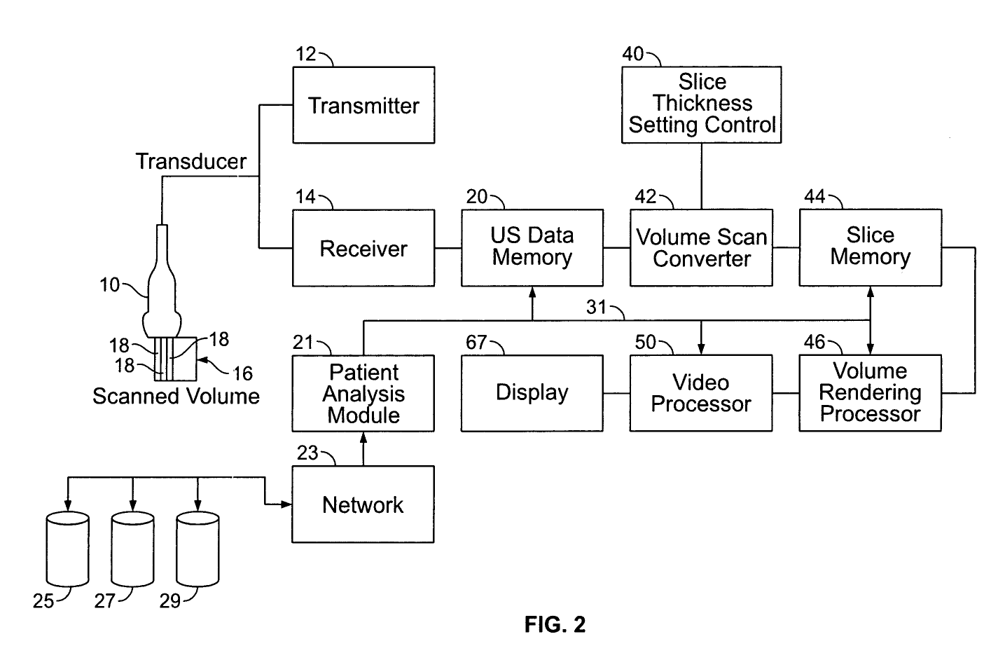

FIG. 2 illustrates an ultrasound system formed in accordance with another embodiment of the present invention. The system includes a probe 10 connected to a transmitter 12 and a receiver 14. The probe 10 transmits ultrasonic pulses and receives echoes from structures inside of a scanned ultrasound volume 16. Memory 20 stores ultrasound data from the receiver 14 derived from the scanned ultrasound volume 16. The volume 16 may be obtained by various techniques (e.g., 3D scanning, real-time 3D imaging, volume scanning, 2D scanning with transducers having positioning sensors, freehand scanning using a Voxel correlation technique, 2D or matrix array transducers and the like).

The position of each echo signal sample (Voxel) is defined in terms of geometrical accuracy (i.e., the distance from one Voxel to the next) and ultrasonic response (and derived values from the ultrasonic response). Suitable ultrasonic responses include gray scale values, color flow values, and angio or power Doppler information.

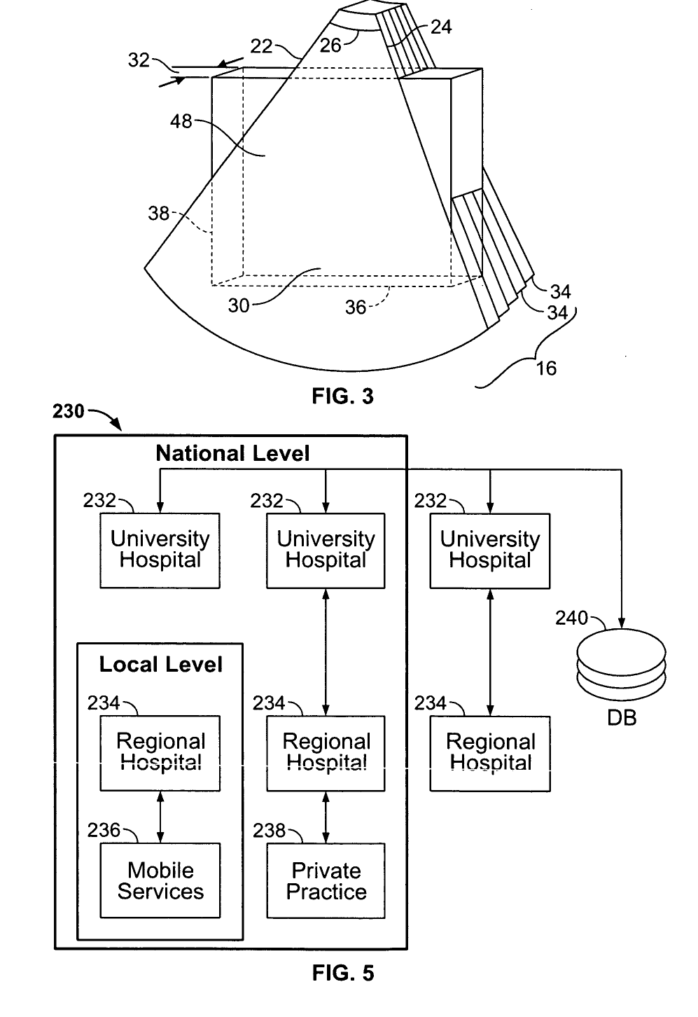

FIG. 3 illustrates a real-time 4D volume 16 acquired by the system of FIG. 1 in accordance with one embodiment. The volume 16 includes a sector shaped cross-section with radial borders 22 and 24 diverging from one another at angle 26. The probe 10 electronically focuses and directs ultrasound firings longitudinally to scan along adjacent scan lines in each scan plane and electronically or mechanically focuses and directs ultrasound firings laterally to scan adjacent scan planes. Scan planes obtained by the probe 10 (FIG. 2), are stored in memory 20 and are scan converted from spherical to Cartesian coordinates by the volume scan converter 42. A volume comprising multiple scan planes is output from the volume scan converter 42 and stored in the slice memory 44 as rendering box 30 (FIG. 3). The rendering box 30 in the slice memory 44 is formed from multiple adjacent image planes 34.

The rendering box 30 may be defined in size by an operator to have a slice thickness 32, width 36 and height 38. The volume scan converter 42 may be controlled by the slice thickness control input 40 to adjacent the thickness parameter of the slice to form a rendering box 30 of the desired thickness. The rendering box 30 designates the portion of the scanned volume 16 that is volume rendered. The volume rendering processor 46 accesses the slice memory 44 and renders along the thickness 32 of the rendering box 30.

During operation, a 3D slice having a pre-defined, substantially constant thickness (also referred to as the rendering box 30) is acquired by the slice thickness setting control 40 (FIG. 2) and is processed in the volume scan converter 42 (FIG. 2). The echo data representing the rendering box 30 may be stored in slice memory 44. Predefined thicknesses between 2 mm and 20 mm are typical, however, thicknesses less than 2 mm or greater than 20 mm may also be suitable depending on the application and the size of the area to be scanned. The slice thickness setting control 40 may include a rotatable knob with discrete or continuous thickness settings.

The volume rendering processor 46 projects the rending box 30 onto an image portion 48 of an image plane 34 (FIG. 3). Following processing in the volume rendering processor 46, the pixel data in the image portion 48 may pass through a video processor 50 and then to a display 67.

The rendering box 30 may be located at any position and oriented at any direction within the scanned volume 16. In some situations, depending on the size of the region being scanned, it may be advantageous for the rendering box 30 to be only a small portion of the scanned volume 16.

The functionality provided by the diagnostic equipment may vary. For example, the diagnostic equipment may be afforded one or more of the following capabilities:

a. Angle independent volume flow measurement as described in U.S. Pat. No. 6,535,836;

b. High spatial and temporal resolution as described in SSP 6,537,217;

c. Real-time 3D (4D) capabilities as described in U.S. Pat. No. 6,450,962;

d. Adjusting operation parameters as described in SSP 6,542,626 and U.S. Pat. No. 6,478,742;

e. Transesophageal probe-based ultrasound, as described in U.S. Pat. No. 6,494,843 and U.S. Pat. No. 6,478,743;

f. Harmonic and sub-harmonic coded excitation as described in U.S. Pat. No. 6,491,631, U.S. Pat. No. 6,487,433, and U.S. Pat. No. 6,478,741;

g. B-mode and Doppler Flow imaging as described in U.S. Pat. No. 6,450,959; and

h. ECG gated image compounding as described in U.S. Pat. No. 6,447,450.

The patents cited in items a through h above are expressly hereby incorporated herein in their entireties.

The diagnostic equipment, such as the ultrasound system 100, is afforded functionality that assists the HC provider to diagnose at least certain pathologies, even when the HC provider is not specialized in such area or does not have significant past experience with the pathology. The HC provider may be a technician, nurse, general practice doctor, and the like. The ultrasound system 100 or other equipment is provided with sufficient state of the art technology to obtain data sets that have high spatial and/or temporal resolution of the patient anatomy. The resolution is dependent in part on the modality (e.g. CT, PET, MR, ultrasound) and in part on the type of diagnostic assistance to be provided (e.g. tumor detection, analysis of fetus health, cardiology studies, general radiology diagnostics, brain tumor/biopsy detection or treatment).

The ultrasound system 100 is further provided with the capability to analyze the new patient's data set to identify and measure certain physiologic parameters. For example, the identification may include detection of the AV-plane of the heart and the like. The measurement may be for the following:

a. tissue velocity or tissue strain rate or derived measurements based on combining such measurements from various anatomical locations in the heart and various timings in the cardiac cycle;

b. time integrations of either tissue velocity or strain rate at selected anatomical location for a subset of the cardiac cycle in order to measure anatomical location for a subset of the cardiac cycle in order to measure tissue motion, tissue synchronicity or strain;

c. heart wall thickness and wall thickening between end diastole and end systole;

d. motion and contraction patterns including velocity profiles and strain rate profiles for selected anatomical locations and subsets of the cardiac cycle;

e. the cardiac rhythm including arrhythmias measured by for instance ECG or tissue velocity or strain rate profiles;

f. organ size and or shape measured in either 2D planes or 3D volumes;

g. comparison of organ size and shape between end diastole and end systole in both 2D planes and 3D volumes including ejection fraction computations;

h. detection of temporal subsections of the cardiac cycle such as systole, diastole, IVC, IVR, E-wave, diastases and A-wave and measurements of parameters or patterns relative to these events; and

i. detection of landmarks and motion patters for these landmarks such as the mitral ring in either 2D planes or 3D volumes.

The ultrasound system 100 may be joined to a decision/routing network 124 and/or a database 128 at link 126 to perform quantitative automated analysis of the physiology parameters for the new patient as explained hereafter. The system of FIG. 2 also includes patient analysis module 21 that communicates with a network 23 and at least one of the data memory 20, slice memory 44 and volume rendering processor 46. The patient analysis module 21 obtains new patient data over link of bus 31 from one of the data memory 20, slice memory 44, video processor 50, and volume rendering processor 46.

Optionally, another memory may be added to store new patient images by one or both of the volume rendering processor 46 and video processor 50, which memory may be accessed by the patient analysis module 21 to obtain the new patient images. Alternatively, the patient analysis module 21 may be removed entirely and then functions and the responsibility thereof performed by one of a master controller (not shown) in the system, video processor 50 and volume rendering processor 46. In this alternative embodiment, link 31 is directly connected to the network 23.

The patient analysis module 21 interfaces with network 23 to obtain past patient data sets stored in one or more of databases 25, 27, and 29. The past patient data may constitute new data, partially processed data, patient images and the like. The databases 25, 27, and 29 may be located at one or different geographic locations or within a common or healthcare network. The databases 25, 27, and 29 may also store common or different types of patient data. For example, database 25 may store ultrasound patient data or images, while databases 27 and 29 store MR and CT patient data or images.

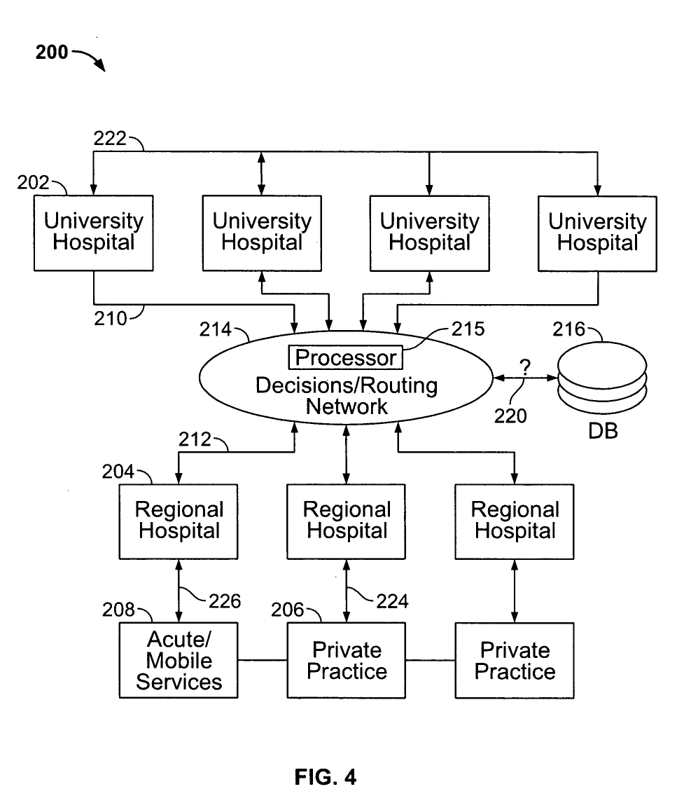

FIG. 4 illustrates a healthcare network 200 that includes various types of healthcare facilities, such as university hospitals 202, regional hospitals 204, private practices 206 and mobile services 208. Clinics may be considered private practices or mobile services 206 and 208. In the illustrated embodiment of FIG. 4, the university hospitals 202 and regional hospitals 204 communicate over network links 210 and 212, with a decision/routing network 214. The decision/routing network 214 accesses and manages a patient database 216 through database link 220. The university hospitals may communicate with one another over link 222 and the private practices and mobile services 206 and 208 may communicate with regional hospitals over links 224 and 226 respectively. The links 210, 212 and 220-226 may represent internet links, dedicated intranets and any other communications network link.

Diagnostic equipment, such as the ultrasound systems shown in FIGS. 1 and 2, may be provided at one or more of the hospitals 202 and 204, private practices 206 and mobile services 208. Optionally, the diagnostic equipment may be shared or shuttled between multiple sites. The diagnostic equipment is used by a physician, a technician, a nurse or the like to examine a patient. Advantageously, the diagnostic equipment may be utilized at a primary healthcare provider by a person who is not necessarily a specialist or exceptionally trained in the usage of such diagnostic equipment, such as the ultrasound systems of FIGS. 1 and 2.

Once an examination is obtained, select patient data is conveyed over the corresponding link (210, 212, 224 and/or 226) until reaching the decision/routing network 214. In the embodiment of FIG. 4, the decision/routing network 214 accesses a database 216, obtain past patient data sets for previously examined patients. In the embodiment of FIG. 4, the decision/routing network 214 may include a host processor or controller 215 that analyzes the current patient information received over links 210 generates a solution or diagnosis and returns the solution or diagnosis to the appropriate healthcare provider at the originating one of hospitals 202 and 204, private practices 206 or mobile services 208. Optionally, the access to knowledge in the database 216 may be provided or controlled by the diagnostic equipment. Further, the database 216 may be embedded or provided on-board the diagnostic equipment. Optionally, the database 216 may store past patient data sets organized and/or catalogued based on pathology type, severeness of a pathology, key patient characteristics that indicate a particular pathology basic patient characteristics (e.g., age, sex, weight, disease type, etc.), and types of anatomic samples that may be obtained for a given type of diagnostic equipment or that are indications of a particular pathology.

By way of example only, the diagnostic equipment may constitute an ultrasound system provided at a private practice 206 of a primary healthcare provider. The primary healthcare provider may image a patient with the ultrasound equipment and request a diagnosis of a particular pathology from the decision/routing network 214. Examples of pathologies to be diagnosed are coronary artery disease, likelihood of heart failure, congenital heart disease, valvular diseases and the like.

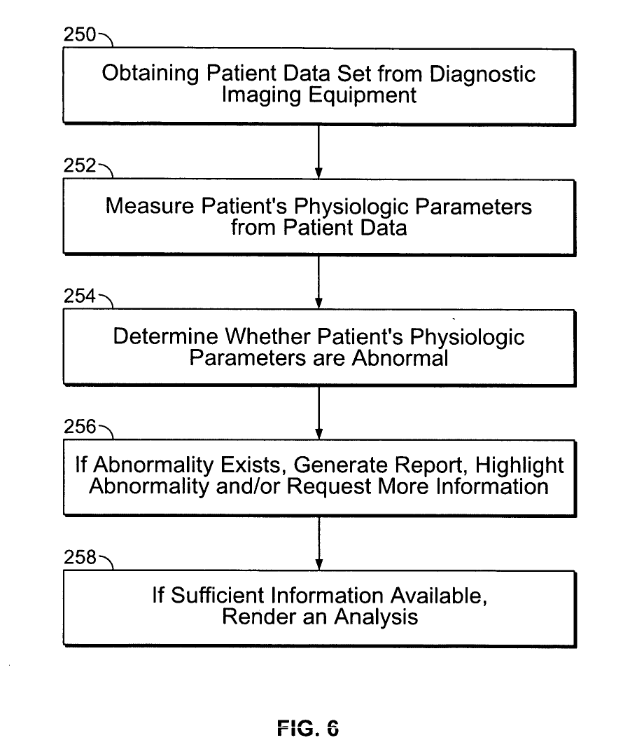

FIG. 5 illustrates an alternative healthcare network 230 that may span internationally. The healthcare network 230 may include university hospitals 232 and regional hospitals 234, mobile services 236 and private practices 238. In one example, a regional hospital 234 may be linked to a mobile service 236 at a local level. Alternatively, a private practice 238 may be linked with a regional hospital 234 and in turn linked with a university hospital 232 at a national level. Even internationally, regional and university hospitals 234 and 232, respectively, may be linked. The university hospitals 232 in turn access a database 240 which may store a library of past patient information.

The new and past patient information may be stored and transferred in a variety of formats in the examples of FIGS. 1 through 5. For example, the raw patient data may be stored within databases FIGS. 1 through 5. Alternatively, the databases patient data volumes or slices forming images resulting from the raw patient data. As a further alternative, the databases may store values for certain physiologic parameters measured from the patient data and/or patient images, where the physiologic parameter is used by physicians to detect and diagnose specific pathologies. FIG. 6 sets forth an exemplary flowchart of an automated analysis that may be performed by any of processor 116 (FIG. 1), patient analysis module 21 (FIG. 2), and processor 215 (FIG. 4). At 250, the patient is examined. At 252, the patients physiologic parameters are automatically identified and measured from the patient data. For example, in echocardiography, at 252, the ultrasound system 100 may automatically identify and measure the AV-plane within an image of the patient's heart. The AV-plane is identified, by locating the apex and boundary of the ventricle. Then, systolic and diastolic measurements of the heart may be obtained. Alternatively, the boundary of the ventricle may be identified and based thereon the dimensions measured of the ventricle or of the ventricle wall thickness. Other automated measurements include tissue velocity imaging to obtain systolic and diastolic waves, transitions in systolic, length of period, e-wave, heart size and shape, and the like.

At 254, the ultrasound system may identify an abnormality directly or, alternatively, send the patient information to a remote processor (e.g., processor 215 in FIG. 4) that, in turn, performs the identification. In one embodiment, the patient's physiologic parameters are compared with physiologic parameters of previously examined patients stored as data sets in a database. The determination at 254 may be a threshold determination based on a comparison of measured parameters with standard acceptable values for the physiologic parameters (stored on the network 215 or locally at the ultrasound system 100).

If no standard acceptable value exists or the patient's physiologic parameters do not clearly exceed accepted values, then at 254 the measured values for the new patient data may be compared to values for the same parameters for past patient data. If an abnormal condition exists, several actions may be taken (step 256). For example, a report for a doctor may be created. Alternatively, images of the patient may be modified to highlight the abnormality (e.g. color coding the image or the surrounding indicia describing the patient). The quantitative analysis may conclude that additional information is needed, such as additional scans of the patient (e.g. different views, additional heart cycles). Additional information may be needed from the HC provider (patient data) or from a different modality (e.g. a prior CT scan, prior MR scan, etc.). The quantitative analysis may conclude that sufficient patient information is available from the current patient to render an analysis (step 258). The analysis may include a diagnosis of the pathology or alternatively indicate that the patient should be referred to a specialist and the like.

Diagnostic imaging in primary HC affords the HC provider with additional information early in the patient examination process. The HC provider is afforded more information unique to the patient's circumstances. A parametric structure or scheme is used that is easy to analyze and for which automated instructions may be provided. Patient specific information is automatically captured by the diagnostic equipment and in one embodiment the HC provider may be walked through a “cookbook” type process to arrive at a solution. For example, the AV-plane of a heart image may be used in numerous studies of the heart. Once the AV-plane is detected, it can be used to monitor the heart cycle, among other thing, measurement of the heart wall thickness allows automatic diagnosis of hypertrophy.

In an alternative embodiment, an on-line network may be provided that permits primary HC providers to interact in real-time or off-line with specialists. The specialist may review the physiologic measurements and/or images while the patient is at the HC provider's office. Alternatively, the HC provider may send the physiologic measurements and/or images to the specialists one day and receive the diagnosis the next day. Optionally, a call center may be established where HC providers may send the physiologic measurements and images for real-time review and analysis.

In certain embodiments, a diagnostic network is provided that accesses a database(s) containing diagnostic information regarding other patients. The diagnostic information includes similar parameters to those measures for the new patient. The source of the data may be ultrasound, x-ray, MRI CT or PET images. The data may constitute raw scan data, processed data sets, resultant images or the values of the associated physiologic parameters as measured from images of prior patients. The database(s) may store a collection of patient studies for an entire hospital or HC network.

The diagnostic network may search one or more databases for similar pathologies and return to the HC provider, patient information for one or more similar studies. The database and/or response may include comments suggesting actions to be taken (e.g. further analysis or treatment). The database may also include known acceptable levels for the measured and other physiologic parameters.

In the event that the patient information is contained in an image, the diagnostic network may analyze the image and compare it to patient images from the database for matches or similar characteristics. The comparison may be based on statistical analysis, measurements, anatomic landmarks, etc. By way of example, in a Doppler analysis, a landmark may be identified in an image and a Doppler spectrum obtained at that landmark. The diagnostic network may then compare the landmark and Doppler spectrum to those of prior patients. In the event that the database includes measurements for the prior patients, the diagnostic network may transfer these measurements to the HC provider or join such measurements with the new patient's images.

Optionally, the diagnostic equipment may perform classification and/or identification based on the physiologic measurements. The classification (e.g. optimize frequency, etc. for arterial blood flow). The measurement may identify to the anatomy (e.g. which heart valve) and suggest the type of anatomy to the HC provider. This measurement may be useful to ensure that the HC provider acquires each type of scan desired for a particular study (e.g. when measuring the size and weight of a fetus, a series of measurements are taken from different anatomical structures). The diagnostic equipment may also highlight features to the HC provider that are unique to a current patient when such features are not found in the database (e.g. a new combination of values for a particular set of physiologic parameters).

The term “controller” as used throughout is intended to be more general then a single processor or group of parallel processors, for instance, the controller may comprise one or multiple computers, processors, CPU's or other devices located remote from the diagnostic equipment or “distributed” between the diagnostic equipment and the decision/routing network 214. The term “distribute” signifies that certain functions of the controller may be performed by and at the diagnostic equipment, while other functions of the controller may be performed by and at a host processor of the decision/routing network 214. For example, the diagnostic equipment may include a local control sub-sections that performs initial analysis of new patient data with respect to one or more physiologic parameters to obtain a patient value(s) for the physiologic parameter(s). The decision/routing network 214 may include a remote control sub-section that utilizes the results of the initial analysis of the new patient data. For instance, the remote control sub-section may compare the patient value(s) for the new patient data with past patient data. Alternatively, the remote control sub-section may compare new patient data directly with past patient data.

Optionally, the diagnostic equipment, controller and/or the decision/routing network may perform searches of the content of the past patient data, such as images, curves, landmarks and other anatomic features. The past patient images, curves, etc. may be searched based on new patient data to locate substantially matching content. For instance, new and past patient images may be compared to locate matching images in the past patient data. Matches may be identified when select features of a past patient image satisfy or fall within limits or other criteria of corresponding features of the new patient image(s).

While the invention has been described with reference to certain embodiments, it will be understood by those skilled in the art that various changes may be made and equivalents may be substituted without departing from the scope of the invention. In addition, may modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A knowledge-based diagnostic imaging system, comprising:
diagnostic equipment for analyzing a patient to obtain a new patient data set containing at least one of MR data, CT data, ultrasound data, x-ray data, SPECT data and PET data, said diagnostic equipment automatically analyzing said new patient data set;
a database containing past patient data sets for previously analyzed patients, said past patient data sets containing data indicative of physiologic parameters with respect to previously analyzed patients;
a network for interconnecting said diagnostic equipment and said database to support access to said past patient data sets; and
a controller for accessing said database based on said new patient data set.

2. The knowledge-based diagnostic imaging system of claim 1, wherein said diagnostic equipment is an ultrasound system and said new patient data set contains at least one ultrasound image.

3. The knowledge-based diagnostic imaging system of claim 1, wherein said physiologic parameter is for the myocardium and said controller accesses said database based on at least one of an AV-plane, tissue velocity, systolic transition, myocardium period length, hypertrophy, diastolic point, heart size and heart shape.

4. The knowledge-based diagnostic imaging system of claim 1, wherein said controller accesses said database based on at least one of contraction patterns and velocity profiles of the myocardium of the previously analyzed patients.

5. The knowledge-based diagnostic imaging system of claim 1, wherein said diagnostic equipment highlights abnormalities in an image generated from said new patent data set.

6. The knowledge-based diagnostic imaging system of claim 1, wherein said diagnostic equipment compares new and past patient data sets to determine whether additional information is needed.

7. The knowledge-based diagnostic imaging system of claim 1, wherein said controller compares at least one of said past patient data sets to said new patient data set.

8. The knowledge-based diagnostic imaging system of claim 1, wherein said diagnostic equipment includes an ultrasound machine for generating a new patient image from said new patient data set and for identifying said physiologic parameter based on said new patient image.

9. The knowledge-based diagnostic imaging system of claim 1, wherein said diagnostic equipment automatically measures values for said physiologic parameter from said new patient data set.

10. The knowledge-based diagnostic imaging system of claim 1, wherein said new and past patient data sets represent new and past patient images, respectively, said controller identifying matches between said new and past patient images.

11. The knowledge-based diagnostic imaging system of claim 1, said controller further comprising a processor located separate and remote from said diagnostic equipment, said processor comparing said new patient data set to said past patient data sets to identify matches.

12. A method for providing knowledge-based diagnostic imaging, comprising:
analyzing a patient to obtain a new patient data set containing at least one of MR data, CT data, ultrasound data, x-ray data, SPECT data and PET data;
automatically analyzing said new patient data set;
accessing past patient data sets for previously analyzed patients, said past patient data sets containing stored patient values indicative of said physiologic parameter with respect to previously analyzed patients; and
analyzing said past patient data sets of previously analyzed patients based on said new patient data set.

13. The method of claim 12, wherein said analyzing the patient includes obtaining ultrasound images of the patient as said new patient data set.

14. The method of claim 12, wherein said automatically analyzing said new patient data set includes measuring at least one of an AV-plane, tissue velocity, systolic transition, myocardium period length, hypertrophy, diastolic point, heart size and heart shape.

15. The method of claim 12, wherein said past patient data sets contain at least one of contraction patterns and velocity profiles of the myocardium of the previously analyzed patients.

16. The method of claim 12, wherein said analyzing the patient includes comparing said new patient data set to at least one of said past patient data sets.

17. The method of claim 12, wherein said analyzing the patient includes generating a new patient image from said new patient data set and said automatically analyzing includes identifying said physiologic parameter from said new patient image.

18. The method of claim 12, wherein said automatically analyzing includes measuring values for said physiologic parameter from a patient image.

19. The method of claim 12, further comprising highlighting abnormalities in an image generated from said new patient data set.

20. The method of claim 12, further comprising comparing new and past patient data sets and determining whether additional information is needed based on said comparison.

21. A network comprising:
diagnostic equipment for analyzing a patient to obtain new patient images based on at least one of MR data, CT data, ultrasound data, x-ray data, SPECT data and PET data, said diagnostic equipment automatically analyzing a said new patient images;
a database containing past patient images for previously analyzed patients; and
an interconnection between said diagnostic equipment and said database, said database providing past patient images for previously analyzed patients; and
a controller for accessing said past patient images based on said new patient images.

22. The network of claim 21, wherein said diagnostic equipment includes an ultrasound machine.

23. The network of claim 21, wherein said physiologic parameter is for the myocardium and includes at least one of an AV-plane, tissue velocity, systolic transition, myocardium period length, hypertrophy, diastolic point, heart size and heart shape.

24. The network of claim 21, wherein said past patient images contain at least one of contraction patterns and velocity profiles of the myocardium of the previously analyzed patients.

25. The network of claim 21, wherein said diagnostic equipment is located at a primary health care site.

26. The network of claim 21, wherein said diagnostic equipment determines where said physiologic parameter for the new patient is abnormal.

27. The network of claim 21, wherein said diagnostic equipment highlights, in said new patient image, an abnormality.

28. The network of claim 21, wherein said diagnostic equipment determines whether additional information is needed from an operator after comparing said new patient image to said past patient images.
